(19) United States
(12) Patent Application Publication
(10) Pub. No.: US 2004/0010816 A1
(43) Pub. Date: Jan. 15, 2004

(54) Transgenic poa grasses

(76) Inventors: Lisa Lee, Marysville, OH (US); Phillip Popham, Marysville, OH (US); Brad Berg, Research Triangle Park, NC (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/380,359

(22) PCT Filed: Sep. 18, 2001
(86) PCT No.: PCT/US01/42194

(51) Int. Cl. A01H 1/00; C12N 15/82; A01H 5/00

(52) U.S. Cl. 800/278; 800/320

(57) ABSTRACT

The invention provides transgenic plant tissue of the genus Poa (preferably Poa pratensis—Kentucky bluegrass—and Poa pratensis×Poa arachnifera—Texas hybrid) that stably expresses heterologous DNA (e.g., for resistance to herbicides such as glyphosate), produced by the transformation of novel embryogenic calli. The invention also comprises embryogenic calli that are particularly susceptible to transformation and regeneration as well as methods for using such calli to produce transgenic plants of the invention.

This application claim the benefit of priority of U.S. provisional application 60/233,592, filed Sep. 18, 2000.

### BACKGROUND OF THE INVENTION

1. Field of the Invention

The invention relates to the field of transgenic plants, plant cell cultivation, plant transformation methods and, in particular, to Poa calli particularly suitable for transformation, methods of making the calli, and transgenic Poa plants and methods of making them.

2. Summary of the Related Art

With over $580 million in retail sales per year, the turfgrass seed market is second only to hybrid seed corn. A principal use of turfgrass seeds is for golf course, of which there are over 14,000 in the U.S. alone with about 300 new courses constructed annually. Each new construction requires 60,000 lbs. of turfgrass seed. Furthermore, intensive management programs are required to maintain the quality and appearance of the courses and to protect them from. weeds and plant diseases. Increasingly, the industry is turning to biotechnology to produce herbicide- and disease-resistant turfgrasses. Lee, Plant Science 115, 1 (1996), provides a general review of turfgrass biotechnology.

Kentucky bluegrass (Poa pratensis) is among the more popular turfgrasses. Plant regeneration in callus cultures of Kentucky bluegrass was reported by van der Valk et al., Plant Cell Reports. 7, 644. (1989), using mature seeds and immature inflorescences as explants. This group also reported improvement of tissue culture response of seed derived callus cultures though gelling agent and abscisic acid (Van Ark et al., Plant Cell, Tissue and Organ Culture 27, 275 (1991)) and benzyladenine (van der Valk et al., Plant Cell Tissue and Organ Culture 40, 101 (1995)).

Dibble et al. reported transformation of seed-derived callus of Kentucky bluegrass variety Glade with the bar and gus genes by particle bombardment. (“Transgene Expression in Kentucky Bluegrass: GUS and BAR”, American Society of Agronomy, 1999, Abstract, p. 160). Selection using bialaphos was done at the plantlet stage.

U.S. Pat. No. 5,948,956 discloses a method a producing transgenic monocotyledonous plants by inserting a foreign genetic material directly into a node segment of a stem of the plant and thereafter subjecting the node segment to conditions sufficient to permit regeneration of the node segment into a plantlet. The process is reported to be especially useful for producing transgenic ryegrasses, fescues and turfgrasses, such as St. Augustinegrass, creeping bentgrass, Kentucky bluegrass, and the like.

The non-selective herbicide, glyphosate (N-phosphonomethyl-glycine) (the active ingredient in ROUNDUP® herbicide (Malik et al., BioFactors 2, 17 (1989)), has proven one of the most safe and effective herbicides for agricultural purposes. Attempts to create glyphosate-resistant plants through the over-expression of wild-type and mutant forms of EPSPS in transgenic plants have met with varied degrees of success. Much attention has focused on the generation and characterization of novel EPSPS mutants created at the site of glyphosate binding to identify those forms that were both highly glyphosate-tolerant and still bound the PEP substrate comparably to the wild-type EPSPS (Padgette et al., J. Biol. Chem. 266, 22364 (1991); T. Ruff et al., Plant Physiol. 96, 94 (1991)). Other studies have identified the critical importance of directing the glyphosate-tolerant EPSPS proteins to the plastid (della-Cioppa et al., Bio/Technology 5, 579 (1987) (“della-Cioppa II”)).

In 1992, Barry and co-workers (Barry et al. in, “Biosynthesis And Molecular Regulation Of Amino Acids In Plants” (B. K. Singh et al. [ed.], Am Soc. Plant Physiologists, Rockville, Md. (1992)) described the isolation and characterization of EPSPS from Agrobacterium sp. strain CP4. The CP4 EPSPS exhibited very favorable biochemical parameters, namely high glyphosate tolerance and tight binding for PEP, which strongly suggested that expression of the cp4 EPSPS gene in transgenic plants might lead to high levels of glyphosate tolerance. This prediction has been borne out as the cp4 EPSPS gene has been introduced into soybean nucleus to create highly glyphosate-tolerant soybeans (Padgette et al., Crop Science 35, 1451 (1995)), which are now commercially available to farmers as ROUNDUP® READY® soybeans. Zhou and colleagues (Zhou et al., Plant Cell Rep. 15, 159 (1995)) have demonstrated that bombardment with the cp4 EPSPS gene, under control of the duplicated CaMV 35S promoter (Kay et al., Science 236, 1299 (1987)), into non-photosynthetic embryogenic callus resulted in nuclear expression of the bacterial enzyme and permitted the recovery of glyphosate-resistant, transgenic wheat plants.

U.S. Pat. No. 5,554,798, “Fertile Glyphosate-resistant transgenic corn plants,” discloses and claims fertile transgenic Zea mays plants containing an isolated heterologous DNA construct encoding EPSP synthase. There is no description in this maize patent on glyphosate selection at all, it is all using hygromycin selection. Resistance of transgenic plants to hygromycin was provided by the expression of hph gene coding hygromycin phosphotransferase (HPH).

### SUMMARY OF THE INVENTION

The present invention comprises transgenic and herbicide resistant plant tissues of the genus Poa. In a preferred embodiment, the invention comprises transgenic Poa plant tissue that is herbicide resistant, preferably glyphosate resistant. Among the preferred transgenic turfgrass plant tissues of the invention are Kentucky bluegrass (Poa pratensis), and Texas hybrid (Poa pratensis L.×Poa arachnifera Torr.). Such plant tissues are resistant to the herbicide glyphosate, preferably at at least 2× (more preferably 4×) levels. In a preferred embodiment, the Kentucky bluegrass and Texas hybrid plants tissues of the invention are transformed with the cp4 gene and express the CP4 protein.

The invention also comprises Poa embryogenic callus tissue that is a particularly useful for transformation and regeneration. Many media composition combinations were tested to obtain the type of embryogenic callus cultures according to the invention that are transformable and regenerable after herbicide selection to obtain transgenic Poa plants.

The invention further comprises a method for producing the transgenic Poa plant tissue and embyogenic callus tissue of the invention.

The foregoing merely summarizes certain aspect of the invention and is not intended, nor should it be construed, as limiting the invention in any manner.

### BRIEF DESCRIPTION OF THE DRAWINGS

FIGS. 1-3 display embryogenic callus cultures according to the invention.

### DETAILED DESCRIPTION OF THE INVENTION

In a first aspect, the present invention comprises transgenic Poa (preferably Kentucky bluegrass—Poa pratensis) and Texas hybrid (Poa pratensis×Poa arachnifera) plant tissue. As described below, the present invention also comprises a novel Poa callus tissue that is particularly well suited for transformation and regeneration which uniquely makes possible for the first time the production of transgenic Poa. Transgenic Poa according to the invention can comprise a wide variety of exogenous genes that express the corresponding proteins at levels sufficient to impart the corresponding characteristic phenotype.

A preferred phenotype is glyphosate resistance, which can. be imparted by genes encoding a modified EPSPS enzyme, such as the cp4 gene (Barry et al., supra), the aroA gene (della-Cioppa II, supra), Class II EPSPS genes (e.g., U.S. Pat. No. 5,633,435), GA21 mutant gene (used in, e.g., ROUNDUP® resistant corn) (WO 95/06128), any other genes encoding a glyphosate-resistant EPSPS enzyme, genes encoding the GOX enzyme (which is a glyphosate oxidoreductase; U.S. Pat. No. 5,776,760), or any other genes encoding enzymes conferring resistance to glyphosate.

Other genes and their corresponding phenotypes contemplated by the invention are the bar gene (glufosinate, bialaphos resistance), nptII (Kanamycin, G418 resistance), and hph (hygromycin resistance).

In one embodiment of this aspect of the invention, the transgenic Poa tissue is herbicide resistant (preferably glyphosate resistant). As used herein, a plant tissue that is “herbicide resistant” (and variations thereof, e.g., “glyphosate resistant”) is one that is able to withstand contact with the minimum amount of herbicide that would kill a similar, untransformed (or wild-type) plant tissue of the same species. It is a routine. matter for the one of ordinary skill in the art to determine the minimal amount of herbicide that would kill a non-transformed (wild-type) plant tissue. Preferably, the plant tissue according to the invention can withstand at least twice the concentration (denoted as “2×”) of herbicide typically applied in field applications; more preferably the transgenic plant tissue of the invention can withstand up to amounts of about 4×. For the herbicide glyphosate, about 2 quarts of 41% glyphosate solution per acre are typically used (which is equivalent to 20 gallons of 2.5% ROUNDUP® ULTRA (41% glyphosate) solution per acre). For hard to control plants such as dandelions, about 4-5 quarts of 41% glyphosate solution per acre are typically used.

As used herein, “tissue” and “plant tissue” mean single cells as well as multicellular tissues such as calli, leaves, stems, roots, stolons, rhizomes, inflorescences and whole plants.

Also encompassed with the invention are seeds that germinate into plant tissue that expresses an exogenous protein that confers a particular phenotype (preferably herbicide (resistance and most preferably glyphosate resistance).

In a preferred embodiment, a Poa plant tissue is transformed with the cp4 gene (Barry et al., supra) and expresses the CP4 protein at levels sufficient to confer glyphosate resistance. In other embodiments, the glyphosate-resistant Poa according to the invention is transformed with other genes whose, expression product imparts glyphosate resistance on the transformed plant, as described above.

Glyphosate-resistant transgenic Poa plant tissue according to the invention expresses the protein encoded by the transgene at levels sufficient to impart glyphosate (e.g., ROUNDUP®) resistance on the plant tissue. These plants are useful, for example, because they will provide lawncare givers (e.g., homeowners and golf course superintendents) the advantage of removing weeds with ROUNDUP® herbicide without adversely affecting their lawns, fairways, and greens.

The transgenic Poa according to this aspect of the invention can be made with the callus tissue according to the second aspect of the invention as described in the fourth aspect of the invention.

In a second aspect, the invention provides non-transformed Poa callus tissue that is particularly susceptible to, and therefore useful for transformation and mutagenesis. Many media composition combinations were tested to obtain the type of embryogenic callus cultures that are transformable and regenerable after selection to obtain transgenic Poa plants. Thus, in a third aspect, the invention comprises a method of generating such calli.

The callus tissue of the invention is generally obtained when it is very young, within from about one month to about four of having been established. The advantage of such calli is that they have a very high regeneration frequency, higher than callus cultures maintained for longer periods of time. The calli useful in the invention can be obtained as follows.

Mature seeds are first sterilized to remove bacteria from their surface. Methods of sterilization are well known to those skilled in the art and any standard method can be used. For example, mature seeds can be treated with a sterilizing agent (e.g., Chlorox, which can be used at about 50% concentration) and tween-20 (e.g., 0.5%) for 5-30 minutes and rinsed with sterile water.

Surface sterilized seeds can be imbibed in sterile water at 4° C. overnight before plating on callus initiation medium. Callus initiation medium contains MS (Murashige and Skoog, 1962) salts and vitamins, sucrose (preferably 2-3%, more preferably, 3%), auxin(s), gelling agent(s), optionally, cytokinins. Preferably, the auxin is 2,4-D in a concentration of at least 0.1 mg/l, but preferably in a concentration of 1 to 4 mg/l, and more preferably, 1 or 2 mg/l 2,4-D. Although cytokinins are not essentially, they are preferably used in a concentration of 0.1 to 2 mg/l and include such cytokinins as 6benzyladenine (6-BAP), thiadiazuron (ADZ), and kinetin, etc., 0.2 to 2 mg/l 6-BAP and 0.2 to 2 mg/l TDZ are more preferred. Preferably the gelling agent is present in concentrations of 0.2 to 0.3%. Suitable gelling agents include phytagel, GELRITE®, and gelgro. Embryogenic callus cultures that are suitable for transformation are selected according to the following criteria:

a) light yellow to white in color and showing no signs of advance necrosis (i.e., brown or black color);

b) the consistency/texture should be rather dry and relatively friable (i.e., easily crumbled or pulverized) and not wet, slimy, or fibrous;

c) small projections, or embryoids, should be easily identifiable on the outer surfaces of the colony.

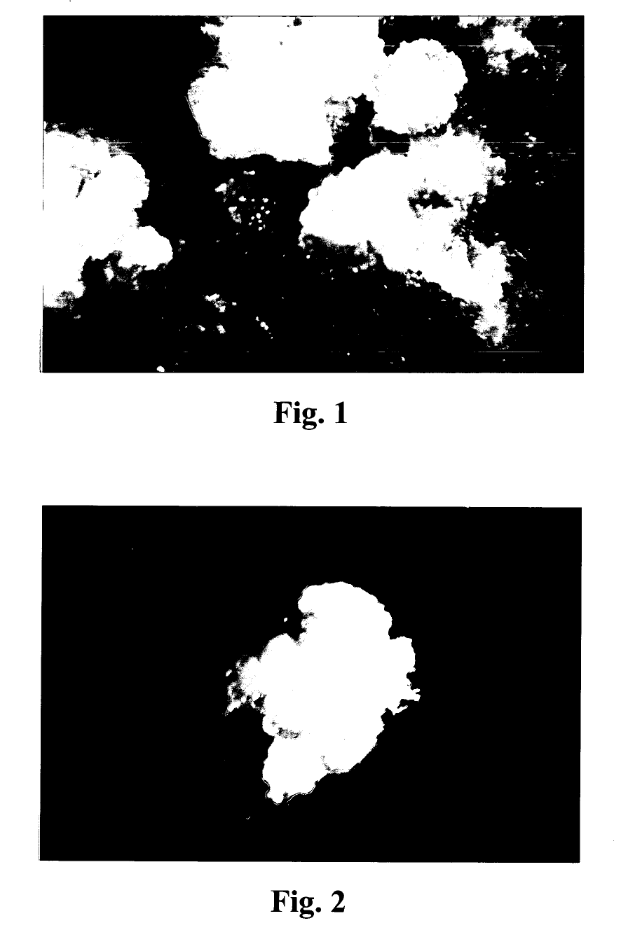

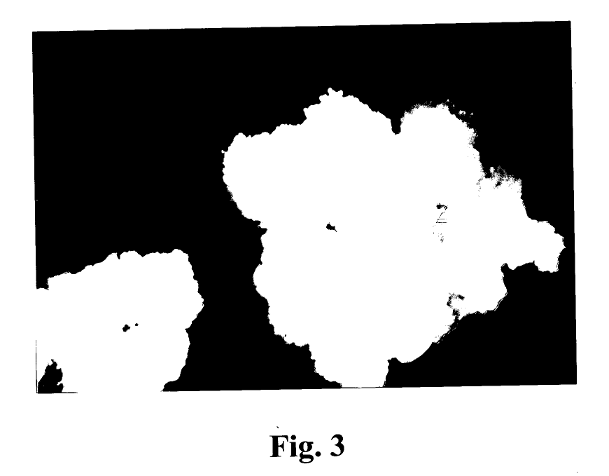

Such calli are generally obtained in approximately 3 to 20 weeks after callus initiation. Careful observation under a dissecting scope is very useful for selection. The embryogenic callus that are maintained for a short time only have a fast growing rate and high regeneration frequency and are particularly well suited as target tissue shortly after they are established. Poa calli according to this aspect of the invention are shown in FIGS. 1-3. We have unexpectedly found that calli that are cultured for more than about 24 weeks are generally unsuitable for transformation and regeneration.

In a fourth aspect, the invention comprises a method of making transgenic plant tissue according to the invention from Poa calli according to the invention. In this aspect the method comprises:

a) transforming embryogenic Poa calli of the invention with a gene (preferably the cp4 gene whose expression product imparts glyphosate resistance on transformed plant tissue);

b) selecting transformed calli (preferably on glyphosate-containing medium);

c) regenerating the selected transformed calli into whole plants.

The success of the presently claimed invention depends upon the selection of appropriate calli for transformation.

Transgenic Poa tissue is obtained by pretreating callus cultures according to the invention with maintenance medium with the addition of mannitol and/or sorbitol (either from 0.2 M to 0.6 M mannitol or sorbitol or, preferably, combinations of 0.1 to 0.3 M mannitol and 0.1 to 0.3M sorbitol, more preferably, 0.1 M mannitol and 0.1 M sorbitol, 0.2 M mannitol and 0.2 M sorbitol, or 0.3 M mannitol and 0.3 M sorbitol) for 4 hours to overnight. Gold particles are coated with DNA following the protocol from Sanford et al., Methods Enzymol. 217, 483 (1992) and coated DNA microprojectiles are bombarded into embryogenic callus cultures using the gene gun. The DNA can also be introduced into these callus cultures via other transformation methods such as protoplast transformation (Lee et al., Crop Sci. 36, 401-406 (1996)), Agrobacterium-mediated delivery, silicon carbide fibers, electroporation of intact tissues, electrophoresis and microinjection (Songstad et al., Plant Cell, Tissue Organ Cult., 40, 1-15 (1995)).

In practice, the nucleic acid constructs comprise not only the herbicide resistance-conferring selectable marker gene, but various control elements as well. Such control elements will preferably include, but are not limited to promoters (e.g., CaMV 35S promoter) and a ribosome binding site (RBS) positioned at an appropriate distance upstream of the translation initiation codon to ensure efficient translation initiation. Where the transgene does not confer a readily selectable phenotype, the nucleic acid constructs for transformation will further comprise a gene that permits easy selection of transformed tissue.

Any of the constructs successfully used in the references cited in the Background of the Invention section that teach nuclear transformation to achieve glyphosate resistance can be used in the present invention. Those skilled in the art will be able to prepare a variety of suitable constructs. A preferable construct is one containing a CaMV 35S promoter or a rice actin promoter with a cp4 gene from Agrobacterium sp. and nos transcriptional termination.

One week after transformation (e.g., by bombardment), selection is initiated. Selection can be conducted using art recognized protocols appropriate for the transgene employed. Many marker genes are know in the art for selection of transgenic cells, tissues, organs or individuals from their wild-type counterparts. Bowen B. A., “Markers for plant gene transfer,” in: Transgenic Plants. Vol. 1, pp. 89-123 (Kung, S. D.; Wu, R, eds. San Diego: Academic Press; 1993) describes 55 effective or potential selective agent/marker gene combinations representing 40 unique selectable, lethal or assayable genes. For calli transformed with a gene imparting glyphosate resistance, for example, selection is begun with between 0.1 and 2 mM glyphosate for 2-4 weeks then either raised between 0.1 mM to 2 mM glyphosate or maintained between 0.1 to 2 mM glyphosate for 2-4 weeks. After 4-8 weeks on selection medium, embryogenic callus cultures are regenerated in the presence of 0.1 to 0.2 mM glyphosate.

Methods of regeneration of the selected calli into whole plants are well known to those skilled in the art and any standard method can be used. Vasil, J. Plant Physiol. 128, 193-218 (1987); Bhaskaran and Smith, Crop Sci. 30, 1328-1336 (1990). For example, the selected resistant calli can be regenerated on a MS basal medium with the incorporation of any of the cytokinins (e.g., BAP, kinetin, ZR, which can be used in the range of 0.1 to 3 mg/l concentration). Regenerated plantlets are rooted in the presence of 0.1 to 0.2 mM glyphosate and transplanted to soil in greenhouse.

Herbicide resistant Poa plants according to the invention can also be made without using transgene insertion. According to this aspect of the invention Poa calli or tissues are mutagenized and subsequently regenerated under selection of herbicide to produce herbicide resistant plants. Either directed mutagenesis (e.g., Mathern and Hake, Genetics, 147(1), 305 (1997)) or random mutagenesis (e.g., Koebner and Hadfield, Genome, 44(1), 45 (2001)) can be utilized to genetically alter wild type Poa. Preferably, a Poa callus according to the invention is subject to mutagenesis and subject to selection of a herbicide (preferably glyphosate or glufosinate).

In another aspect, the invention comprises transgenic and herbicide resistant Poa plants made according to the methods of the invention.

Among the Poa species contemplated for use in all aspects of the invention are those presented in Table D1 (the “major” Poa species, which are preferred for use in the invention) and Table D2:

TABLE D1

| Latin name | Common Name |
|---|---|
| Poa ampla Merr. | Big bluegrass |
| Poa annua L. | Annual Bluegrass |
| Poa arachnifera Torr. | Texas bluegrass |
| Poa bulbosa L. | Bulbous bluegrass |
| Poa compressa L. | Canada Bluegrass |
| Poa nemoralis L. | Wood bluegrass |
| Poa palustris L. | Fowl bluegrass |
| Poa pratensis L. | Kentucky bluegrass |
| Poa supine | — |
| Poa trivialis L. | Rough bluegrass |

TABLE D2

| Latin Name | Common Name |
|---|---|
| Poa alpina L. | Alpine bluegrass |
| Poa alsodes A. Gary | — |
| Poa arctica R. Br. | Artic bluegrass |
| Poa arida Vasey | Plains bluegrass |
| Poa arropurpurea Scribn. | — |
| Poa autumnalis Muhl. | — |
| Poa bigelovii Vasey and Scribn. | Bigelow Bluegrass |
| Poa bolanderi Vasey | — |
| Poa canbyi (Scribn.) Piper | Canby bluegrass |
| Poa chaixii Vill. | — |
| Poa chapmaniana Scribn. | — |
| Poa confines Vasey | — |
| Poa curta Rydb. | — |
| Poa curtifolia Scribn. | — |
| Poa cusickii Vasey | Cusick bluegrass |
| Poa cuspidate Nutt. | — |
| Poa douglasii Nees | — |
| Poa epilis Scribn. | Skyline bluegrass |
| Poa fendleriana (Steud.) Vasey | Mutton grass |
| Poa fernoldiana Nannf. | — |
| Poa fibrata Swallen | — |
| Poa glauca Vahl | — |
| Poa glaucantha Gaudin | — |
| Poa glaucifolia Scribn. and Williams | — |
| Poa gracillima Vasey | Pacific bluegrass |
| Poa howellii Vasey and Scribn. | Howell Bluegrass |
| Poa interior Rybd. | Inland bluegrass |
| Poa involuta Hitchc. | — |
| Poa juncifolia Scribn. | Alkali bluegrass |
| Poa kelloggii Vasey | — |
| Poa languida Hitchc. | — |
| Poa laxiflora Buckl. | — |
| Poa leibergii Scribn. | Leiberg bluegrass |
| Poa leptocoma Trinius | Bog bluegrass |
| Poa lettermani Vasey | — |
| Poa longiligula Scribn. and Williams | Longtongue Mutton grass |
| Poa macrantha Vasey | — |
| Poa macroclada Rybd. | — |
| Poa marcida Hitchc. | — |
| Poa montrevansi Kelso | — |
| Poa napensis Beetle | — |
| Poa nervosa (Hook.) Vasey | Wheeler bluegrass |
| Poa nevadensis Vasey ex Scribn. | Nevada bluegrass |
| Poa occidentalis Vasey | New Mexican bluegrass |
| Poa paludigena Fern. and Wieg. | — |
| Poa pattersoni Vasey | Patterson bluegrass |
| Poa paucispicula Scribn. and Merr. | — |
| Poa pringlei Scribn. | — |
| Poa reflexa Vasey and Scribn. | Nodding bluegrass |
| Poa rhizomata Hitchc. | — |
| Poa rupicola Nash | Timberline bluegrass |
| Poa saltuensis Fern. and Wieg. | — |
| Poa scabrella (Thurb.) Benth. ex Vasey | Pine bluegrass |
| Poa secunda Presl | Sandberg bluegrass |
| Poa stenantha Trin. | — |
| Poa sylvestris A. Gray | — |
| Poa tracyi Vasey | — |
| Poa unilateralis Scribn. in Vasey | — |
| Poa vaseyochloa Scribn. | — |
| Poa wolfii Scribn. | — |

Preferred Poa pratensis varieties contemplated for use in all aspects of the invention are Abbey, America, Ascot, A960-328, Chateau, Coventry, Fairfax, H94-301, Limousine, Midnight, Merit, Touchdown, Unique, and Victa.

Using the same protocol as described herein (and/or routine modifications thereof adapted for the particular species and/or gene), embryogenic calli of any Poa turfgrass species can be prepared and transformed to yield regenerable transformed plant tissue expressing the protein encoded by the gene and exhibiting the corresponding phenotype.

The following examples are provided for illustrative purposes only, and are not intended, nor should they be construed as limiting the invention (as described in detail above) in any manner. Those of ordinary skill in the art will appreciate that modifications and variations of the following examples can be made without exceeding the scope of the invention as set forth above and in the claims below. All patents, patent applications, and other publications are hereby incorporated by reference in their entirety.

### EXAMPLES

### Example 1

### Production of Glyphosate Resistant Transgenic Kentucky Bluegrass and Texas Hybrid

Tissue

Embryogenic callus cultures derived from surface sterilized mature turfgrass seeds were used for transformation. Mature seeds were treated with 50% Chlorox with 0.5% tween-20 for 30 minutes, rinsed with sterile water for 4 times and stored at 4° C. overnight before plating seeds on callus initiation medium. The callus initiation medium contained MS (Murashige and Skoog, 1962) salts and vitamins, 2 or 3% sucrose, a combination of 2,4-D between 0.5 to 2 mg/l and cytokinins such as 6-BAP and TDZ between 0.1 to 2 mg/l and 0.2 to 0.3% phytagel (or gelgro or gelrite) as gelling agent. Approximately 4 to 24 weeks after callus initiation, embryogenic callus cultures were selected and used for bombardment.

DNA Used for Transformation

One DNA construct used for bombardment contained the cp4 gene, which confers glyphosate resistance when expressed in plant cells. The plant transformation vector is p25496:

| | | | | | |
|---|---|---|---|---|---|
| CaMV | cp4 | nos 3′ | Act1 | cp4 | nos 3′ |

It encodes two copies of cp4EPSPS genes optimized for plant transformation. The rice actin 1 promoter element (Act1) and the 35S cauliflower mosaic virus (CaMV) promoter with the duplicated enhancer region were used to drive expression of the cp4EPSPS genes. The 3′ end of the nopaline synthase gene (nos 3′) from Agrobacterium tumefaciens T-DNA provides the polyadenylation sites for the cp4 EPSPS gene.

DNA Delivery

The DNA was introduced into the embryogenic callus cultures via a particle bombardment process. In general, embryogenic callus cultures were pretreated with maintenance medium with the addition of mannitol and sorbitol (i.e., 0.1 M mannitol and 0.1 M sorbitol, 0.2 M mannitol and 0.2 M sorbitol, or 0.3 M mannitol and 0.3 M sorbitol) for 4 hours to overnight. Gold particles were coated with DNA following the protocol from Sanford et al., supra, and coated DNA microprojectiles were bombarded into embryogenic callus cultures using the gene gun.

Selection Process

Selection was initiated with 0.5 to 2 mM glyphosate for 2-4 weeks, then either raised from 0.5 to 2 mM glyphosate or maintained between 0.5 to 2 mM glyphosate.

Plant Regeneration

Bombarded embryogenic callus cultures were regenerated after 4-8 weeks on selection in the presence of 0.2 mM to 0.1 mM glyphosate. Regenerating plantlets were rooted in Phytatrays (Sigma) in the presence of between 0.2 mM to 0.1 mM glyphosate. Rooted regenerated plants were transplanted to soil in the greenhouse. Table 1A shows the varieties of Kentucky bluegrass plants and number of each variety that were grown in the greenhouse and, after plants were established in the greenhouse, planted and grown in the field. Table 1A also shows the results of glyphosate treatment of 115 plants regenerated from the fourteen Kentucky bluegrass varieties. A total of 83 plants (out of 96 tested) survived 128 oz/A (i.e., 5× rates) ROUNDUP® greenhouse spray tests.

Table 1A also shows the results of Southern blot hybridization assays of about 63 plants. All showed the incorporation of the cp4 gene into the plant genome.

TABLE 1A

| | | # of plants | # of plants confirmed by |
|---|---|---|---|
| | # of plants | survived/# of | Southern/# of plants analyzed |
| Variety | produced | plants sprayed | by Southern |
| Abbey | 14 | 10/14 | 10/10 |
| America | 18 | 16/17 | 2/2 |
| Ascot | 17 | 15/15 | 8/8 |
| A960-328 | 6 | 1/1 | 1/1 |
| Chateau | 9 | 8/9 | 8/8 |
| Coventry | 1 | 1/1 | 1/1 |
| Fairfax | 12 | 9/11 | 10/10 |
| H94-301 | 2 | NA | NA |
| Limousine | 8 | 0/1 | NA |
| Midnight | 1 | NA | NA |
| Merit | 7 | 6/7 | 6/6 |
| Touchdown | 2 | 1/2 | 1/1 |
| Unique | 2 | 2/2 | 2/2 |
| Victa | 16 | 14/16 | 14/14 |
| Total | 115 | 83/96 | 63/63 |

These experiments were repeated as described above except that Texas hybrid (Poa pratensis×Poa arachnifera) was employed in place of Kentucky bluegrass. (For a discussion of Texas hybrid, see Read, International Turfgrass Society Research Journal 9,202-205 (2001).) The results are reported in Table 1B:

TABLE 1B

| | | # of plants | # of plants confirmed by |
|---|---|---|---|
| | # of plants | survived/# of | Southern/# of plants analyzed |
| Variety | produced | plants sprayed | by Southern |
| HB-124 | 17 | 12/12 | 8/8 |
| HB-125 | 6 | 4/5 | 4/4 |
| HB-126 | 20 | 17/18 | 14/14 |
| HB-127 | 7 | 2/3 | 2/2 |
| HB-130 | 24 | 16/18 | 11/11 |
| HB-131 | 22 | 17/22 | 9/9 |
| HB-337 | 3 | 1/1 | NA |
| Total | 99 | 70/79 | 48/48 |

These experiments were repeated again on both Poa pratensis, Poa pratensis ×Poa arachnifera but this time using several single-copy CP4 constructs:

| Name | Construct | | |
|---|---|---|---|
| p39050 | mCaMV | cp4 | p17 3′ |
| p25487 | CaMV | cp4 | nos 3′ |

p39050: encodes one copy of cp4 EPSPS gene driven by a modified 35S cauliflower mosaic virus (CaMV) promoter and the 3′ end of wheat heat shock protein 17 providing the polyadenylation site).

p25487: encodes one copy of the cp4 EPSPS gene driven by the 35S cauliflower mosaic virus (CaMV) promoter with the duplicated enhancer region and the 3′ end of the nopaline synthase gene (nos 3′) from Agrobacterium tumefaciens T-DNA providing the polyadenylation site).

The results for Kentucky bluegrass are provided in Table 1C(i)-(ii):

TABLE 1C(i)
construct: p39050

| | | # of plants | # of plants confirmed by |
|---|---|---|---|
| | # of plants | survived/# of | Southern/# of plants analyzed |
| Variety | produced | plants sprayed | by Southern |
| America | 3 | 2/2 | — |

TABLE 1C(ii)
construct: p25487

| | | # of plants | # of plants confirmed by |
|---|---|---|---|
| | # of plants | survived/# of | Southern/# of plants analyzed |
| Variety | produced | plants sprayed | by Southern |
| America | 1 | 1/1 | — |
| Unique | 2 | 1/1 | — |

The results for Texas hybrid are give in Table 1D(i)-(ii):

TABLE 1D(i)
construct: p39050

| | | # of plants | # of plants confirmed by |
|---|---|---|---|
| | # of plants | survived/# of | Southern/# of plants analyzed |
| Variety | produced | plants sprayed | by Southern |
| HB-127 | 3 | 2/2 | — |
| HB-130 | 3 | 3/3 | — |
| HB-131 | 13 | 7/7 | 1/1 |

TABLE 1D(ii)
construct: p25487

| | | # of plants | # of plants confirmed by |
|---|---|---|---|
| | # of plants | survived/# of | Southern/# of plants analyzed |
| Variety | produced | plants sprayed | by Southern |
| HB-125 | 5 | 2/2 | — |

Effect of Medium on Callus Formation

Seeds from Abbey and Ascot varieties of Kentucky Bluegrass were subjected to a variety of supplemented Murashige and Skoog media and observed for callus formation. Table 2 presents the results (no. of seeds produced embryogenic callus cultures divided by no. of seeds tested).

TABLE 2
Effect of different media on embryogenic callus
formation of Kentucky bluegrass varieties Abbey and Ascot

| Medium^{a} | Abbey | Ascot |
|---|---|---|
| MS + 1 mg/l 2,4-D | 70/1250 (5.6%) | 3/200 (1.5%) |
| MS + 0.1 mg/l 2,4-D and 1 mg/l | 20/750 (2.7%) | 27/1150 (2.3%) |
| BAP | | |
| MS + 2 mg/l 2,4-D and 150 mg/l | 93/2000 (4.65%) | 5/2850 (0.2%) |
| asparagine | | |
| MS + 5 mg/l 2,4-D and 150 mg/l | 0/100 (0%) | 0/100 (0%) |
| asparagine | | |
| MS + 5 mg/l 2,4-D | 4/250 (1.6%) | 0/250 (0%) |
| MS + 2 mg/l 2,4-D and 2 mg/l TDZ | 59/500 (12%) | 1/500 (0.2%) |
| MS + 2 mg/l 2,4-D and 0.2 mg/l | 12/500 (2.4%) | 7/500 (1.4%) |
| TDZ | | |
| MS + 2 mg/l 2,4-D and 2 mg/l BAP | 36/300 (12%) | 43/500 (8.6%) |
| MS + 2 mg/l 2,4-D and 0.2 mg/l | 31/345 (9%) | 32/300 (11%) |
| BAP | | |

We claim:

1. A transgenic Poa plant tissue.

2. The Poa plant tissue according to claim 1, wherein the plant tissue is herbicide resistant.

3. The Poa plant tissue according to claim 2, wherein the herbicide is glyphosate.

4. The Poa plant tissue according to claim 2, wherein the herbicide is glufosinate.

5. An herbicide resistant Poa plant tissue.

6. The Poa plant tissue according to claim 5, wherein the herbicide is glyphosate.

7. The Poa plant tissue according to claim 5, wherein the herbicide is glufosinate.

8. The plant tissue according to claim 3 or 6, wherein the plant tissue is resistant to at least 2× glyphosate.

9. The plant tissue according to claim 3 or 6, wherein the plant tissue is resistant to at least 4× glyphosate.

10. The plant tissue according to claim 3 wherein the plant tissue is transformed with and expresses a protein encoded by a gene selected from the group consisting of cp4, aroA, Class II EPSPS genes, GA21 mutant, and a gene encoding the GOX enzyme.

11. The plant tissue according to claim 10, wherein the gene is cp4.

12. The plant tissue according to claim 1, wherein the plant tissue is transformed with and expresses a protein encoded by a gene selected from the group consisting of bar, nptII, and hph.

13. The Poa plant according to any one of claims 1-7 or 10-12, wherein the plant is a Poa pratensis plant.

14. The Poa plant according to any one of claims 1-7 or 10-12 , wherein the plant is a Poa pratensis L. ×Poa arachnifera Torr plant.

15. The Poa plant of claim 13, wherein the plant is of a variety selected from the group consisting of Abbey, America, Ascot, A960-328, Chateau, Coventry, Fairfax, H94-301, Limousine, Midnight, Merit, Touchdown, Unique, Apollo, and Victa.

16. A Poa seed, wherein the seed germinates into a Poa plant tissue according to any one of claims 1-7 or 10-12.

17. A Poa seed, wherein the seed germinates into a Poa plant tissue according to claim 8.

18. A Poa seed, wherein the seed germinates into a Poa plant tissue according to claim 9.

19. A Poa seed, wherein the seed germinates into a Poa plant tissue according to claim 13.

20. A Poa seed, wherein the seed germinates into a Poa plant tissue according to claim 14.

21. A Poa seed, wherein the seed germinates into a Poa plant tissue according to claim 15.

22. A method of producing a Poa embryogenic callus susceptible to transformation and regeneration, the method comprising:
a) culturing sterilized Poa seeds on callus initiation medium for about 3 to 24 weeks; and
b) selecting a callus that is light yellow to white in color, friable, and having embryoids on the outer surfaces.

23. The method according to claim 22, wherein the seeds are Poa pratensis seeds.

24. The method according to claim 22, wherein the seeds are Poa pratensis L.×Poa arachnifera Torr. seeds.

25. The method according to claim 23, wherein the Poa pratensis seeds are from a variety selected from the group consisting of Abbey, America, Ascot, A960-328, Chateau, Coventry, Fairfax, H94-301, Limousine, Midnight, Merit, Touchdown, Unique, Apollo, and Victa

26. A Poa embryogenic callus susceptible to transformation and regeneration produced by the method of any one of claims 22 through 25.

27. A method of producing transgenic Poa callus, the method comprising:
a) transforming an embryogenic callus or calli according to claim 26 with an exogenous gene;
b) selecting one or more transformed calli expressing a protein encoded by the exogenous gene.

28. The method according to claim 27, wherein the exogenous gene encodes a protein that confers herbicide resistance to the callus.

29. The method according to claim 28, wherein the exogenous gene encodes a protein that confers glyphosate resistance to the callus.

30. The method according to claim 29, wherein the gene is selected from the group consisting of cp4, aroA, Class II EPSPS genes, GA21 mutant, and a gene encoding the GOX enzyme.

31. The method according to claim 30, wherein the gene is cp4.

32. The method according to claim 27, wherein the gene is selected from the group consisting of bar, nptII, and hph.

33. The method according to claim 27, wherein the Poa is Poa pratensis or Poa pratensis L.×Poa arachnifera Torr.

34. The method according to claim 33, wherein the Poa is a Poa pratensis variety selected from the group consisting of Abbey, America, Ascot, A960-328, Chateau, Coventry, Fairfax, H94301, Limousine, Midnight, Merit, Touchdown, Unique, Apollo, and Victa

35. The method according to claim 29, wherein the Poa is a Poa pratensis variety selected from the group consisting of Abbey, America, Ascot, A960-328, Chateau, Coventry, Fairfax, H94-301, Limousine, Midnight, Merit, Touchdown, Unique, and Victa.

36. A method of producing a non-callus, transgenic, Poa plant tissue, method comprising regenerating the Poa-callus according to claim 35.

37. The method according to claim 31, wherein the Poa is a Poa pratensis variety selected from the group consisting of Abbey, America, Ascot, A960-328, Chateau, Coventry, Fairfax, H94-301, Limousine, Midnight, Merit, Touchdown, Unique, and Victa.

38. A method of producing a non-callus, transgenic, Poa plant tissue, method comprising regenerating the Poa-callus according to claim 27.

39. A method of producing a non-callus, transgenic, Poa plant tissue, method comprising regenerating the Poa-callus according to claim 28.

40. A method of producing a non-callus, transgenic, Poa plant tissue, method comprising regenerating the Poa-callus according to claim 29.

41. A method of producing a non-callus, transgenic, Poa plant tissue, method comprising regenerating the Poa-callus according to claim 30

42. A method of producing a non-callus, transgenic, Poa plant tissue, method comprising regenerating the Poa-callus according to claim 31.

43. A method of producing a non-callus, transgenic, Poa plant tissue, method comprising regenerating the Poa-callus according to claim 32.

44. A method of producing a non-callus, transgenic, Poa plant tissue, method comprising regenerating the Poa-callus according to claim 33.

45. A method of producing a non-callus, transgenic, Poa plant tissue, method comprising regenerating the Poa-callus according to claim 34.

46. A method of producing a non-callus, transgenic, Poa plant tissue, method comprising regenerating the Poa-callus according to claim 35.

47. A method of producing a non-callus, transgenic, Poa plant tissue, method comprising regenerating the Poa-callus according to claim 36.

48. A method of producing a non-callus, transgenic, Poa plant tissue, method comprising regenerating the Poa-callus according to claim 37.

49. A Poa plant tissue produced according to the method of claim 38.

50. A Poa plant tissue produced according to the method of claim 39.

51. A Poa plant tissue produced according to the method of claim 40.

52. A Poa plant tissue produced according to the method of claim 41.

53. A Poa plant tissue produced according to the method of claim 42.

54. A Poa plant tissue produced according to the method of claim 43.

55. A Poa plant tissue produced according to the method of claim 44.

56. A Poa plant tissue produced according to the method of claim 45.

57. A Poa plant tissue produced according to the method of claim 46.

58. A Poa plant tissue produced according to the method of claim 47.

59. A Poa plant tissue produced according to the method of claim 48.
